(19) United States
(12) Patent Application Publication
(10) Pub. No.: US 2021/0065878 A1
(43) Pub. Date: Mar. 4, 2021

(54) Data Transmission System for Transmitting and Receiving Medical Information Data and Data Transmission Method Thereof

(71) Applicant: IRM INC., Gyeonggi-do (KR)

(72) Inventor: Seung Wook CHOI, Gyeonggi-do (KR)

(21) Appl. No.: 16/955,973

(22) PCT Filed: Jun. 4, 2020
(86) PCT No.: PCT/KR2020/007248

§ 371 Date: Jun. 19, 2020

(30) Foreign Application Priority Data
Aug. 27, 2019 (KR) 10-2019-0105285

(51) Int. Cl. G16H 30/20; G16H 40/67; G16H 40/20; H04L 29/08

(52) U.S. Cl. CPC G16H 30/20; G16H 40/67; H04L 67/12; H04L 67/1097; G16H 40/20

(57) ABSTRACT

The disclosure provides a data transmission system for transmitting and receiving medical information data and a data transmission method thereof. In more detail, the disclosure provides a data transmission system for transmitting and receiving medical information data using Digital Imaging and Communications in Medicine (DICOM), a standard for a medical information format, and a data transmission method thereof. The disclosure includes: receiving medical information data including unique receiver information; comparing the unique receiver information with a pre-stored mapping table to identify whether there is a medical information storage device corresponding to the unique receiver information; and when there is the medical information storage device corresponding to the unique receiver information, transmitting the medical information data to the identified medical information storage device.

### TECHNICAL FIELD

One or more embodiments relate to a data transmission system for transmitting and receiving medical information data and a data transmission method thereof. In more detail, one or more embodiments relate to a data transmission system for transmitting and receiving medical information data using Digital Imaging and Communications in Medicine (DICOM), a standard for a medical information format, and a data transmission method thereof.

### BACKGROUND ART

With the development of medical technology and communication technology, attempts to converge the medical industry and the communication industry have been actively conducted in recent years. In particular, research is being conducted on a technique for acquiring medical image data including a diagnosis result for a patient and transmitting/receiving the medical image data between spaced devices.

On the other hand, as a medical imaging device (e.g., computer tomography (CT), X-ray, etc.) outputs an image photographed by a digital method rather than an existing analog method, each manufacturer of the medical imaging device has designed the medical imaging device to have a unique image output format. At this time, when using the manufacturer's unique image output format, there has been a problem with compatibility with equipment of other companies.

To solve this problem, American College of Radiology (ACR) and National Electrical Manufacture Association (NEMA) have established DICOM, a standard for medical imaging format.

DICOM has been gradually researched and expanded not only in medical image format but also in the area of all types of medical information data, including communication, transmission, and display of medical image data and medical data that is not an image (e.g., electrocardiogram information, etc.).

Even if DICOM establishes a standard for medical information data, each device cannot communicate with each other when communication protocols between devices do not match. That is, DICOM provides not only the format of medical information data, but also standards for transmission and communication.

Referring to FIG. 1 schematically showing a conventional DICOM standard medical information data format, a DICOM standard medical information data set includes at least one data element (DE), and each data element includes Tag, Length, Value, and Value Representation (VR). Tag indicates what a data element is, Length may be a length of each data element, and Value may be the content of an actual data element.

VR indicates an identifier type of data, and may include Application Entity (AE), Age String (AS), Attribute Tag (AT), Code String (CS), and Decimal string (DS).

In particular, AE is the most basic value for initiating DICOM communication, and may be made to distinguish and recognize DICOM communication partners. AE is also called AE Title, and means a DICOM communication name of a device capable of DICOM communication. When any AE Title of a transmitting/receiving device does not exist or is unknown, DICOM communication cannot be performed.

However, most medical devices from the same manufacturer are often set to the same AE Title. For this reason, medical information data transmitted from one medical institution (e.g., hospital) to a cloud server may be recognized as being obtained from one medical device because the calling AE Title is the same, even if the medical device obtaining medical information data is different.

Conventionally, a gateway device is installed in a medical institution (hospital), and a unique tag is attached to each medical information to be transmitted from each hospital and uploaded to a cloud server.

On the other hand, a TCP port received from the cloud server may be transmitted differently, but there is a limitation that the TCP port of the cloud server cannot be increased indefinitely. In addition, in the past, a method of differently calling AE Title has been attempted, but there has been a problem in that a situation in which the called AE Title overlaps may occur.

### DESCRIPTION OF EMBODIMENTS

### Technical Problem

One or more embodiments include a Digital Imaging and Communications in Medicine (DICOM) data transmission system capable of efficiently transmitting/receiving medical information data between spaced devices by setting unique receiver information for each communication pair, and a data transmission method thereof.

However, this is merely an example, and the scope of the disclosure is not limited thereto.

### Solution to Problem

According to one or more embodiments, a data transmission method includes receiving medical information data including unique receiver information; comparing the unique receiver information with a pre-stored mapping table to identify whether there is a medical information storage device corresponding to the unique receiver information; and when there is the medical information storage device corresponding to the unique receiver information, transmitting the medical information data to the identified medical information storage device.

In addition, the receiving is to receive first medical information data including first unique receiver information from a first medical information acquisition device, the identifying is to compare the first unique receiver information with the pre-stored mapping table to identify whether there is a first medical information storage device corresponding to the first unique receiver information, and the transmitting is to transmit the first medical information data to the first medical information storage device.

In this case, the first unique receiver information is generated based on identification information of the first medical information acquisition device and unique information of the first medical information storage device.

In addition, the receiving is to receive second medical information data second first unique receiver information from a first medical information acquisition device, the identifying is to compare the second unique receiver information with the pre-stored mapping table to identify whether there is a second medical information storage device corresponding to the second unique receiver information, and the transmitting is to transmit the second medical information data to the second medical information storage device.

In this case, the second unique receiver information is generated based on identification information of the first medical information acquisition device and unique information of the second medical information storage device.

Meanwhile, a recording medium according to an embodiment may be a non-transitory computer-readable recording medium having recorded thereon a program for executing the above-described method.

According to one or more embodiments, a data transmission method includes a transceiver configured to receive medical information data including unique receiver information, and a processor configured to compare the unique receiver information with a pre-stored mapping table to identify whether there is a medical information storage device corresponding to the unique receiver information and to control the transceiver to transmit the medical information data to the identified medical information storage device when there is the medical information storage device corresponding to the unique receiver information.

In this case, the transceiver receives first medical information data including first unique receiver information from a first medical information acquisition device, and the processor compares the first unique receiver information with the pre-stored mapping table to identify whether there is a first medical information storage device corresponding to the first unique receiver information and controls the transceiver to transmit the first medical information data to the first medical information storage device.

Also, the first unique receiver information is generated based on identification information of the first medical information acquisition device and unique information of the first medical information storage device.

Also, the transceiver receives second medical information data including second unique receiver information from a first medical information acquisition device, and the processor compares the second unique receiver information with the pre-stored mapping table to identify whether there is a second medical information storage device corresponding to the second unique receiver information and controls the transceiver to transmit the second medical information data to the second medical information storage device.

In this case, the second unique receiver information is generated based on identification information of the first medical information acquisition device and unique information of the second medical information storage device.

According to one or more embodiments, a data transmission system includes a medical information acquisition device, at least one medical information storage device, and a cloud server that receives medical information data including unique receiver information from the medical information acquisition device, compares the unique receiver information with a pre-stored mapping table to identify whether there is a medical information storage device corresponding to the unique receiver information among the at least one medical information storage device, and transmits the medical information data to the identified medical information storage device when there is the medical information storage device corresponding to the unique receiver information.

These and/or other aspects will become apparent and more readily appreciated from the following description of the embodiments, taken in conjunction with the accompanying drawings.

### Advantageous Effects of Disclosure

According to the disclosure as described above, each medical institution may use specifically generated unique receiver information as DICOM standard receiver information (called AE Title) to transmit medical information data to a target device through a cloud server without installing a gateway device.

In addition, according to the disclosure, medical information data may be accurately and automatically transmitted to intended device and system.

However, the scope of the disclosure is not limited to the effect.

### BRIEF DESCRIPTION OF DRAWINGS

FIG. 1 is a schematic view of conventional DICOM standard medical information data format;

FIG. 2 is a system diagram illustrating a medical information data transmission system according to an embodiment;

FIG. 3 is a simple block diagram for explaining components of a cloud server according to an embodiment;

FIGS. 4 and 5 are simple flowcharts for explaining a data transmission method of a cloud server according to an embodiment;

FIG. 6 is a view for explaining in detail that a cloud server according to an embodiment generates a hash value for each communication pair;

FIG. 7 is a diagram illustrating a mapping table according to an embodiment;

FIG. 8 is a sequence diagram illustrating a data transmission method of a data transmission system according to an embodiment; and

FIG. 9 is a diagram illustrating generating and managing hash value information in a cloud server according to an embodiment.

### MODE OF DISCLOSURE

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, various embodiments of the disclosure are described in connection with the accompanying drawings. Various embodiments of the disclosure may have various modifications and various embodiments, and specific embodiments are illustrated in the drawings and related detailed descriptions are described. However, this is not intended to limit the various embodiments of the disclosure to specific embodiments, and should be understood to include all modifications and/or equivalents or substitutes included in the spirit and technical scope of the various embodiments of the disclosure. Similar reference numerals are assigned to similar elements throughout the specification.

It will be further understood that the terms “comprises” and/or “comprising” when used in various embodiments of the disclosure, specify the presence of stated features, integers, steps, operations, elements, and/or components, but do not preclude the presence or addition of one or more other features, integers, steps, operations, elements, components, and/or groups thereof.

In various embodiments of the disclosure, expressions such as “or” include any and all combinations of words listed together. For example, “A or B” may include A, may include B, or may include both A and B.

It will be understood that although the terms “first”, “second”, “third”, etc. may be used herein to describe various components, these components should not be limited by these terms. For example, the above expressions do not limit the order and/or importance of the components, and may be used to distinguish one component from another component.

In the embodiments of the disclosure, terms such as “module”, “unit”, “part”, and the like are used to refer to a component that performs at least one function or operation, and the component may be implemented by hardware or software or a combination of hardware and software. In addition, a plurality of “modules”, “units”, “parts”, and the like may be implemented as at least one processor by being integrated with at least one module or chip, except where each needs to be implemented as individual specific hardware.

It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, various embodiments of the disclosure will be described in detail with reference to the accompanying drawings.

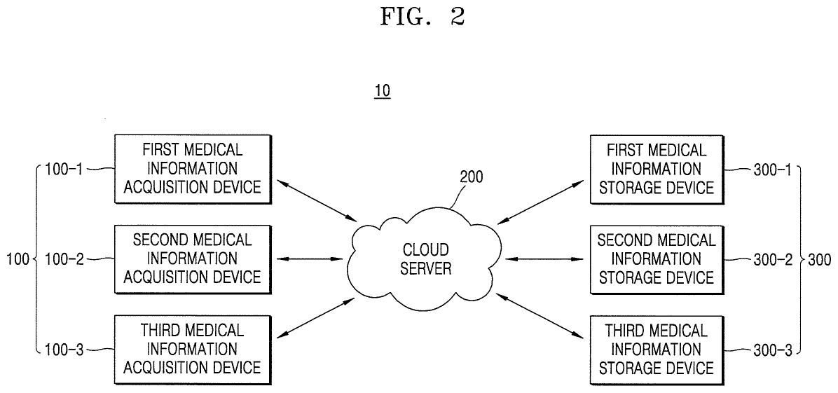

FIG. 2 is a system diagram illustrating a medical information data transmission system according to an embodiment.

Referring to FIG. 2, a system 10 may include a medical information acquisition device 100, a cloud server 200, and a medical information storage device 300. Meanwhile, in the following, medical information data according to the disclosure may include all kinds of data that can be processed in general computing devices such as images, waveforms, and measured values.

In FIG. 2, the medical information acquisition device 100 includes a first medical information acquisition device 100-1, a second medical information acquisition device 100-2, and a third medical information acquisition device 100-3. However, this is only an example, and the medical information acquisition device 100 may include various numbers of medical information acquisition devices according to embodiments. Similarly, the medical information storage device 300 is illustrated as including a first medical information storage device 300-1, a second medical information storage device 300-2, and a third medical information storage device 300-3. However, this is only an example, and the medical information storage device 300 may include various numbers of medical information storage devices according to embodiments.

In the disclosure, the medical information acquisition device 100 refers to all types of devices capable of obtaining and transmitting medical information data including information about a patient. For example, the medical information acquisition device 100 may be one of an X-ray imaging device, a CT imaging device, an MRI device, a PET imaging device, and an OCT imaging device, but is not limited thereto. Also, the medical information acquisition device 100 according to an embodiment may refer to a system including two or more of all the above-described types of device.

In the disclosure, the cloud server 200 may transmit and receive medical information data between each medical information acquisition device 100 and each medical information storage device 300 in the system 10.

The cloud server 200 may connect each medical information acquisition device 100 and each medical information storage device 300 to each other according to a wired/wireless communication protocol. For example, the cloud server 200 may perform various processes such as searching, storing, transmitting, querying, exchanging, and connecting medical information data based on medical information standards such as IHE, DICOM, and HL7 and international standards such as W3C and ISO.

For example, the cloud server 200, when receiving medical information data including sender information (calling AE Title) and receiver information (called AE Title) from each medical information acquisition device 100, may transmit the medical information data to the medical information storage device 300 corresponding to receiver information (called AE Title).

The receiver information (called AE Title) of the disclosure may be that unique receiver information for each communication pair (e.g., hash value information) generated by the cloud server 200 is input. According to an embodiment, the cloud server 200 may generate a hash value based on identification information (calling AE Title) of the medical information acquisition device 100 and unique identifier (UID) of the medical information storage device 300, and may transmit medical information data to the medical information storage device 300 corresponding to a hash value mapped for each communication pair. A detailed description thereof will be provided later below.

In the disclosure, the medical information storage device 300 may be a picture archiving communication system (PACS), an electronic medical record (EMR) system, a radiology information system (RIS), and/or a laboratory information system (LIS), but is not limited thereto.

At this time, the picture archiving communication system (PACS) is a system that may store or transmit a medical image, and refers to a system used by users (e.g., medical professionals such as doctors) to read a stored medical image and perform image diagnosis.

The electronic medical record (EMR) system refers to a system capable of computerizing, inputting, managing, and storing information related to patient care (i.e., medical records). At this time, the medical records refer to all information related to a patient, such as a patient's personal information, medical history, medical condition, medical records, and hospitalization or discharge records.

The radiology information system (RIS) is an analysis and management system for medical images such as X-ray images, computer tomography (CT) images, and magnetic resonance images (MRI), and a system that performs general management functions of radiology, such as analysis report management and imaging reservation management, and management of reference materials for image analysis.

The laboratory information system (LIS) refers to a network established by each laboratory and each department of the hospital. For example, through the laboratory information system, test results are automatically entered in a patient registration system as test items in the laboratory, and the doctor in charge may check the results on a terminal. In most hospitals, the necessary test items are displayed according to the doctor's instructions, so a laboratory pathology department, a diagnostic radiology department, and the like that do not directly deal with patients often do not know what the patient's problem is. Therefore, through the laboratory information system, a user may view patient's records through a terminal in each laboratory and review data necessary for diagnosis.

Meanwhile, one medical information data includes one data element and may be included in zero or more data sets. In other words, the medical information data includes one Application Entity (AE) Title information, and may not be included in a data set or may be included in one or more data sets.

A data set means a set of actual data elements. Data processing in the medical information acquisition device 100 may be performed in units of data sets, and the data sets need to belong to one group. Here, the group may mean a group of physical organizations such as a hospital or a department, or a virtual set of users.

Meanwhile, receiver information (called AE Title) included in each data element of medical information data according to an embodiment may be information corresponding to one group.

That is, the system 10 according to an embodiment may classify at least one medical information data including the same receiver information (or called AE Title) into one data set, and may perform data processing in units of data sets.

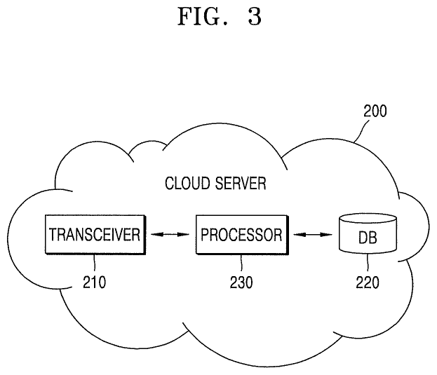

FIG. 3 is a simple block diagram for explaining components of a cloud server according to an embodiment.

Referring to FIG. 3, the cloud server 200 may include a transceiver 210, a database (DB) 220, and a processor 230.

The transceiver 210 may communicate with various types of external devices according to various types of communication methods. The transceiver 210 may include at least one of a Wi-Fi chip, a Bluetooth chip, and a wireless communication chip. The processor 230 may communicate with an external server or various external devices using the transceiver 210. In addition, the transceiver 210 may communicate with external devices through various communication chips such as an NFC chip.

The processor 230 (or a controller) may control the overall operation of the cloud server 200 using various programs stored in a memory (not shown). The processor 230 may include RAM, ROM, a main CPU, first to n interfaces, and a bus. At this time, the RAM, the ROM, the main CPU, the first to n interfaces, and the like may be connected to each other through a bus.

The database 220 is a group of organized data, and is a bundle of common data that can be received from a plurality of devices and operated by storing integrated information. That is, the database 220 may integrate and store medical information data received from a plurality of medical information acquisition devices 100.

According to an embodiment, the processor 230 may store various data received through the transceiver 210 in the database 220. In particular, the processor 230 may store identification information (e.g., AE Title) and unique information (UID) of the medical information acquisition device 100 and the medical information storage device 300 in the database 220. The processor 230 may generate a hash value mapping table based on the identification information (AE Title) and the unique information (UID), or the like, and may control the transceiver 210 to transmit some or all of hash value mapping table information to each of the medical information acquisition devices 100.

According to an embodiment, the processor 230 may determine the first medical information storage device 300 corresponding to first medical information data based on unique receiver information (e.g., a hash value) included in first medical information data received through the transceiver 210 and a mapping table stored in the database 220. Thereafter, the processor 230 may control the transceiver 210 to transmit the first medical information data to the first medical information storage device 300.

The above-described embodiments are only examples, and the processor 230 may be connected to the transceiver 210 and the database 220 to control the transceiver 210 and the database 220 to perform various processes described in the disclosure of the cloud server 200.

Meanwhile, in FIG. 3, the transceiver 210, the database 220, and the processor 230 are illustrated as being included as separate modules in the cloud server 200, but are not limited thereto. In other words, at least two or more components of the cloud server 200 may be implemented as one module.

In addition, although FIG. 3 illustrates that the transceiver 210, the database 220, and the processor 230 are implemented in one server, this is only an example. Each component of the cloud server 200 may be implemented in a different server. For example, when the database 220 is implemented in a separate server, the processor 230 may be connected to the database 220 through the transceiver 210.

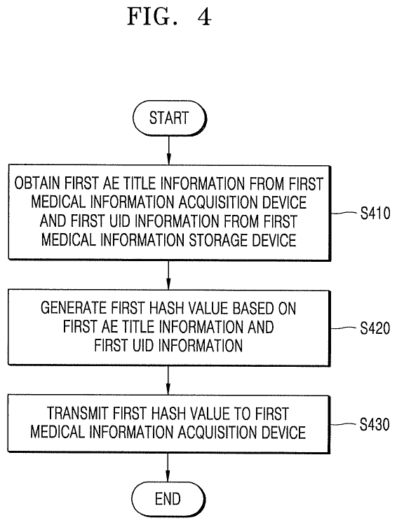

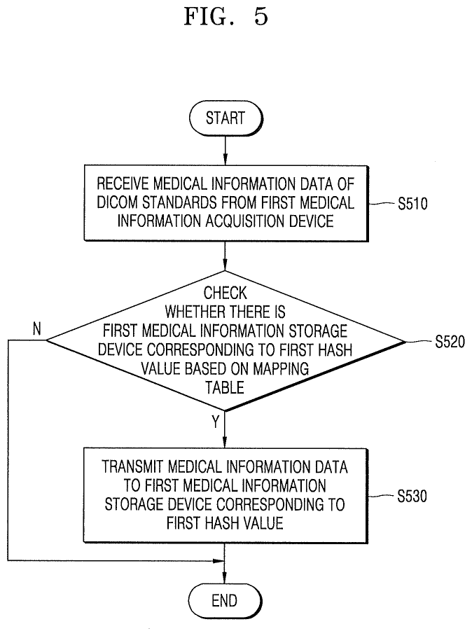

Hereinafter, examples in which the cloud server 200 operates with reference to FIGS. 4 and 5 will be described. FIGS. 4 and 5 are simple flowcharts for explaining a data transmission method of a cloud server according to an embodiment.

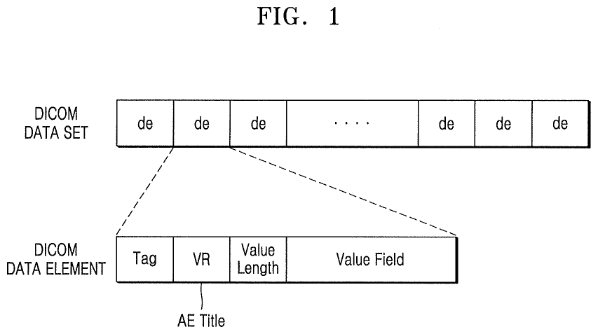

Referring to FIGS. 4 and 5, the data transmission method includes operations that are processed in time series in the cloud server 200 shown in FIG. 1. Accordingly, it can be seen that even if omitted below, the contents described in the entire specification are applied to the data transmission method of the cloud server 200 of FIGS. 4 and 5.

Referring to FIG. 4, in operation S410, the cloud server 200 may obtain first AE Title information from the first medical information acquisition device 100-1 and first UID information from the first medical information storage device 300-1.

At this time, the first AE Title information is the most basic value for initiating DICOM communication, and may refer to a DICOM communication name of the first medical information acquisition device 100-1. Further, the first UID information may mean unique information for identifying the first medical information storage device 300-1.

The above-described AE Title information is only an example of sender identification information, and the identification information may vary according to embodiments such as a host name and a sender IP address. Similarly, the above-described UID information is only an example of receiver unique information, and unique information for identifying the medical information storage device 300 may use a TCP/IP number in addition to UID, but is not limited thereto.

In operation S420, the cloud server 200 may generate a first hash value based on the obtained first AE Title information and the obtained first UID information. As described above, in addition to generating a first hash value based on the first AE Title information and the first UID information, the cloud server 200 may generate a hash value based on at least one of sender AE Title information, a sender Host Name, a sender IP address, receiver UID information, and a receiver TCP/IP number according to an embodiment.

The cloud server 200 of the embodiment of FIG. 4 may input the first AE Title information and the first UID information to a hash function as input values. The hash function may output a hash value of a fixed length based on the input values. Because such a hash function outputs a completely different hash value even if only a portion of the input values is changed, each input value may have a unique hash value.

That is, because the hash function of the disclosure uses AE Title of the first medical information acquisition device 100-1 and UID of the first medical information storage device 300-1 as input values, the hash function outputs a completely different hash value even if only one input value is changed.

A hash function or a hash algorithm of the disclosure may be implemented by an MD algorithm (Message-Digest algorithm) and/or a SHA algorithm (Secure Hash Algorithm). The MD algorithm, especially an MD5 algorithm, provides a 128-bit hash. The SHA algorithm is a hash function that generates a value of 160 bits, and an input value may be a block with a size of 512 bits.

However, the above-described example is only an example, and the hash function or hash algorithm may be implemented through various algorithms such as an SHA-0 algorithm and an SHA-1 algorithm in addition to the SHA-256 algorithm that provides a 160-bit hash.

In operation S430, the cloud server 200 may transmit the generated first hash value to the first medical information acquisition device 100-1. Thereafter, when medical information data is transmitted to the first medical information storage device 300-1, the first hash value may be input to receiver information (called AE Title). This will be further described with reference to FIG. 5.

Referring to FIG. 5, when it is desired to transmit medical data information to the first medical information storage device 300-1, as described above, the first medical information acquisition device 100-1 may transmit medical data to the cloud server 200 by inputting the received first hash value to DICOM standard receiver information (called AE Title) as unique receiver information. At this time, in the disclosure, the unique receiver information may have the same meaning as that of a paired AE Title. In other words, the first medical information acquisition device 100-1 may transmit medical data by inputting a paired AE Title generated in the cloud server 200 to the DICOM standard receiver information (called AE Title).

Meanwhile, according to an embodiment, the first medical information acquisition device 100-1, in addition to a first hash value, may use a derived value generated based on the first hash value as the unique receiver information. In this case, the derived value may mean a value obtained by processing a hash value to increase compatibility with the DICOM standard receiver information (called AE Title).

When receiving medical information data of DICOM standards from the first medical information acquisition device 100-1 in operation S510, the cloud server 200 may determine whether to include a unique first hash value, which means to transmit from the first medical information acquisition device 100-1 to the first medical information storage device 300-1.

In more detail, in operation S520, the cloud server 200 may check whether there is the first medical information storage device 300-1 corresponding to the first hash value based on a mapping table for a hash value.

When it is determined that there is the first medical information storage device 300-1 corresponding to the first hash value in operation S520-Y, in operation S530, the cloud server 200 may transmit medical information data to the first medical information storage device 300-1. On the other hand, when there is no medical information storage device corresponding to the first hash value in operation S520-N, the cloud server 200 does not transmit the received medical information data to a separate medical information storage device.

According to an embodiment, when there is no medical information storage device corresponding to the first hash value in operation S520-N, the cloud server 200 may transmit a signal informing that there is no medical information storage device corresponding to the first hash value to the first medical information acquisition device 100-1. According to another embodiment, the cloud server 200 may transmit a transmission rejection signal including information indicating that data transmission to the first medical information acquisition device 100-1 is rejected.

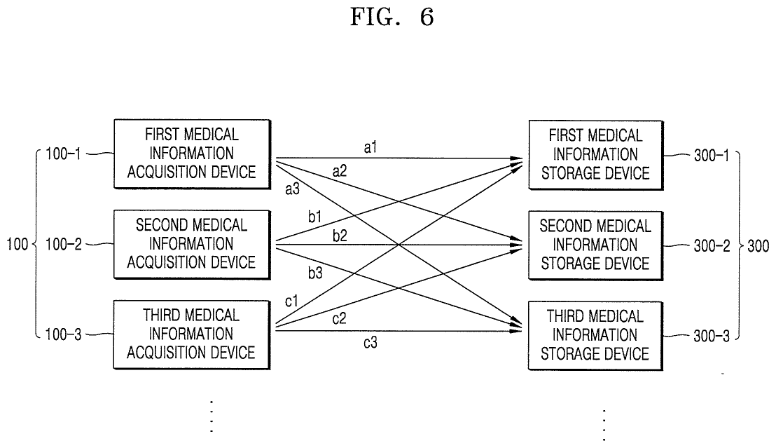

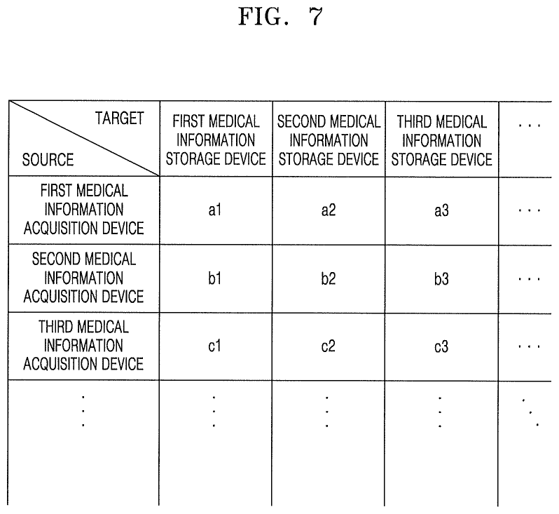

Meanwhile, the mapping table of the disclosure may be a table in which each hash value is mapped to each medical information acquisition device 100 and each medical information storage device 300, the each hash value being generated based on AE Title of each medical information acquisition device 100 and UID of each medical information storage device 300. This will be described in detail with reference to FIGS. 6 and 7.

FIG. 6 is a view for explaining in detail that a cloud server according to an embodiment generates a hash value for each communication pair.

The cloud server 200 of the disclosure may obtain identification information (e.g., the AE Title) about each of the first medical information acquisition device 100-1, the second medical information acquisition device 100-2, and the third medical information acquisition device 100-3, and unique information (e.g., the UID) about each of the first medical information storage device 300-1, the second medical information storage device 300-2, and the third medical information storage device 300-3.

Referring to FIG. 6, the cloud server 200 may generate a first hash value a1 based on first AE Title information about the first medical information acquisition device 100-1 and first UID information about the first medical information storage device 300-1.

In similar, the cloud server 200 may generate a second hash value a2 based on the first AE Title information about the first medical information acquisition device 100-1 and second UID information about the second medical information storage device 300-2. The cloud server 200 may generate a third hash value a3 based on the first AE Title information about the first medical information acquisition device 100-1 and third UID information about the third medical information storage device 300-3. The cloud server 200 may generate a fourth hash value b1 based on second AE Title information about the second medical information acquisition device 100-2 and the first UID information about the first medical information storage device 300-1. The cloud server 200 may generate a fifth hash value b2 based on the second AE Title information about the second medical information acquisition device 100-2 and the second UID information about the second medical information storage device 300-2. The cloud server 200 may generate a sixth hash value b3 based on the second AE Title information about the second medical information acquisition device 100-2 and the third UID information about the third medical information storage device 300-3. The cloud server 200 may generate a seventh hash value c1 based on third AE Title information about the third medical information acquisition device 100-3 and the first UID information about the first medical information storage device 300-1.

The cloud server 200 may generate an eighth hash value c2 based on the third AE Title information about the third medical information acquisition device 100-3 and the second UID information about the second medical information storage device 300-2. The cloud server 200 may generate a ninth hash value c3 based on the third AE Title information about the third medical information acquisition device 100-3 and the third UID information about the third medical information storage device 300-3.

However, this is only an example, and the cloud server 200 may generate a hash value for each communication pair based on identification information (e.g., AE Title) for each of a variety of medical image acquisition devices and unique information (e.g., UID) for each of a variety of medical information storage devices.

FIG. 7 is a diagram illustrating a mapping table according to an embodiment.

Referring to FIG. 7, the cloud server 200 may generate a mapping table to correspond to a source and a target, respectively, based on a plurality of hash values obtained in FIG. 6.

Further, according to an embodiment, the cloud server 200 may generate a derived value based on the obtained hash values. In this case, the derived value may mean a value obtained by processing a hash value to increase compatibility with DICOM standard receiver information (called AE Title). The cloud server 200 may generate a mapping table to correspond to a source and a target, respectively, based on each derived value.

When receiving the DICOM standard medical information data (called AE Title) from the medical information acquisition device 100, the cloud server 200 may check receiver information called AE Title) included in the data.

In this case, the medical information acquisition device 100 that has transmitted medical information data may store hash value information corresponding to each target generated by the cloud server 200. At this time, the medical information acquisition device 100 may obtain and store the hash value information by receiving data including the hash value information from the cloud server 200, but is not limited thereto. The medical information acquisition device 100 may obtain and store the hash value information in various ways as well as the above method.

The medical information acquisition device 100 according to an embodiment may input and transmit unique receiver information (a hash value or a derived value generated based on the hash value) corresponding to a target (medical information storage device) to which medical information data is to be transmitted to the DICOM standard receiver information (called AE Title).

The cloud server 200 may search whether unique receiver information included in the received medical information data exists in the mapping table. When the unique receiver information exists in the mapping table, the cloud server 200 may transmit the received medical information data to the target (medical information storage device) corresponding to the unique receiver information.

For example, when the second medical information acquisition device 100-2 intends to transmit medical information data to the third medical information storage device 300-3, the second medical information acquisition device 100-2 may input the sixth hash value b3, that is, unique receiver information, into the DICOM standard receiver information (called AE Title).

When a device corresponding to unique receiver information (or paired AE Title in this disclosure) included in the DICOM standard medical information data is identified as the third medical information storage device 300-3, the cloud server 200 may transmit the medical information data to the third medical information storage device 300-3.

Meanwhile, when receiver information (called AE Title) included in the received DICOM standard medical information data is not unique receiver information included in the mapping table, the cloud server 200 may transmit a transmission rejection signal including information indicating that data transmission to the medical information acquisition device 100 that has transmitted medical information data is rejected. However, this is only an example, and according to another embodiment, the cloud server 200 may transmit a signal for notifying that target receiver information is unclear.

In the above-described case, the cloud server 200 may transmit a request signal informing the medical information acquiring device 100 that has transmitted the medical information data to check receiver information for a target device.

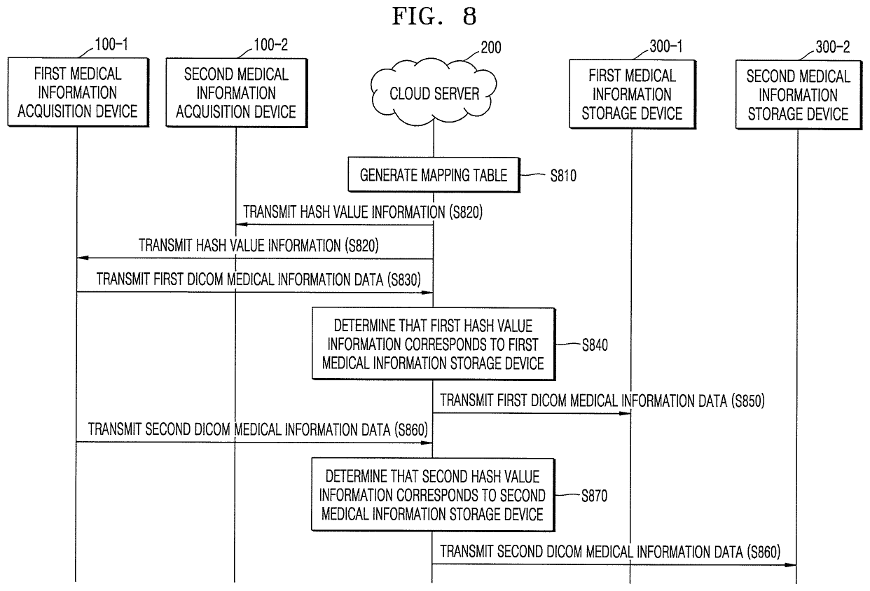

FIG. 8 is a sequence diagram illustrating a data transmission method of a data transmission system according to an embodiment. Hereinafter, for convenience of description, it is assumed that a hash value includes a derived value corresponding to the hash value, and a mapping table generated based on the hash value includes a mapping table generated based on the derived value corresponding to the hash value.

Referring to FIG. 8, as shown in FIGS. 6 and 7, in operation S810, the cloud server 200 may generate a mapping table to correspond to a source and a target, respectively, based on obtained hash values.

In operation S820, the cloud server 200 may transmit hash value information included in the mapping table to the first medical information acquisition device 100-1 and the second medical information acquisition device 100-2. In more detail, the cloud server 200 may transmit hash value information corresponding to each of at least one target to the first medical information acquisition device 100-1, which is a source of medical information data. For example, the cloud server 200 may transmit first hash value information and second hash value information respectively corresponding to the first medical information storage device 300-1 and the second medical information storage device 300-2 to the first medical information acquisition device 100-1. In this case, the first hash value information and the second hash value information may be transmitted to the first medical information acquisition device 100-1 and the second medical information acquisition device 100-2 through data including the hash value information, but are not limited thereto. The first medical information acquisition device 100-1 and the second medical information acquisition device 100-2 may obtain and store the hash value information in various ways in addition to the above method.

Meanwhile, in operation S830, the first medical information acquisition device 100-1 may transmit first DICOM medical information data input with the first hash value information as receiver information to the cloud server 200. In operation S840, the cloud server 200 may check the first hash value information included in the first DICOM medical information data, and may determine that the first hash value information corresponds to the first medical information storage device 300-1. In operation S850, the cloud server 200 may transmit the first DICOM medical information data to the first medical information storage device 300-1.

Likewise, in operation S860, the second medical information acquisition device 100-2 may transmit second DICOM medical information data input with the second hash value information as receiver information to the cloud server 200. In operation S870, the cloud server 200 may check the second hash value information included in the second DICOM medical information data, and may determine that the second hash value information corresponds to the second medical information storage device 300-2. The cloud server 200 may transmit the second DICOM medical information data to the second medical information storage device 300-2.

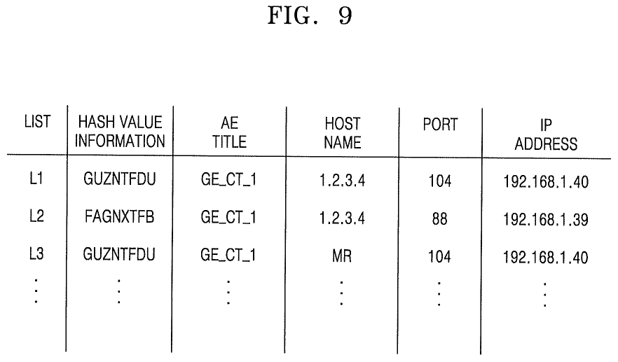

FIG. 9 is a diagram illustrating generating and managing hash value information in the cloud server 200 according to an embodiment.

Referring to FIG. 9, LIST is a list that is named by L1 to Ln in an order in which the cloud server 200 generates hash values, and a hash value information item is a list showing the hash values generated by the cloud server 200 in an order to correspond to LIST items in the same column. AE Title is a list showing an AE Title of the medical information acquisition device 100 that is the basis of hash value information in the same column. Host Name is a list listing names set by a user of the cloud server 200 to correspond to L1 to Ln of LIST in the same column, respectively. Port is a list showing TCP values of the medical information storage device 300 that is the basis of the hash value information in the same column. IP address is a list showing IP values of the medical information storage device 300 that is the basis of the hash value information in the same column.

At this time, an application layer protocol of a TCP/IP stack of the medical information storage device 300 may be distinguished by a unique port number. Accordingly, the cloud server 200 according to an embodiment may collect the unique port number as unique information of the medical information storage device 300, and may generate a hash value based on this. That is, the cloud server 200 of the disclosure may obtain hash value information based on AE Title of the medical information acquisition device 100 and a TCP/IP unique port number of the medical information storage device 300.

In the example of FIG. 9, when comparing columns L1 and L2, AE Title and Host Name are the same as GE_CT_1 and 1.2.3.4, respectively. However, IP addresses of columns L1 and L2 are different from each other as 192.168.1.40 and 192.168.1.39, respectively, and Ports are also different from each other as 104 and 88, respectively. In this case, even if the AE Title and Host Name are the same, the hash value information are different from each other as GUZNTFDU and FAGNXTFB, respectively.

That is, even if the AE Title and Host Name of the medical information acquisition device 100 are the same as GE_CT_1 and 1.2.3.4, respectively, when received information of the medical information storage device 300 (e.g., TCP/IP) are different from each other, the cloud server 200 generates different hash value information. In addition, the cloud server 200 may transmit the different hash value information to the medical information acquisition device 100. In this example, when hash value information included in medical information data received from the medical information acquisition device 100 is GUZNTFDU, the cloud server 200 may transmit data to the medical information storage device 300 having IP address 192.168.1.40 and Port 104, and when the hash value information included in the medical information data received from the medical information acquisition device 100 is FAGNXTFB, may transmit data to the medical information storage device 300 having IP address of 192.168.1.39 and Port of 88.

In the example of FIG. 9, when comparing columns L1 and L3, Host Names are 1.2.3.4 and MR, respectively. However, AE Titles of columns L1 and L3 are the same as GE_CT_1, IP addresses are the same as 192.168.1.40, and Ports are also the same as 104, respectively.

The hash value information of FIG. 9 may have already been generated in the cloud server 200 and stored in the medical information acquisition device 100 based on sender identification information and receiver information.

The medical information acquisition device in columns L1 and L3 may already store unique receiver information (Hash value information GUZNTFDU) generated in pairs for a target including IP address 192.168.1.40/Port 104 as the same source as AE Title GE_CT_1. For example, even if Host Name of a medical information acquisition device in column L3 is changed from 1.2.3.4 to MR, the medical information acquisition device in column L3 stores the hash value information GUZNTFDU to a medical information storage device corresponding to IP address 192.168.1.40/Port 104.

In this case, the medical information acquisition device in columns L1 and L3 may input the hash value information GUZNTFDU to the medical information storage device corresponding to IP address 192.168.1.40/Port 104 in receiver information (called AE Title).

When a hash value included in plural pieces of medical information data received from the medical information acquisition device in columns L1 and L3L1 and L3 is GUZNTFDU, the cloud server 200 may transmit plural pieces of medical information data to the medical information storage device 300 having TCP/IP of IP address 192.168.1.40 and Port 104.

That is, even if the Host Names of senders are different, when hash value information is the same as GUZNTFDU, the cloud server 200 may transmit data from the same source to the same target.

As in the above-described embodiment, because the system 10 according to an embodiment determines whether to transmit data by checking each paired unique receiver information (a hash value and/or a derived value generated based on the hash value) between a transmitting device (e.g., a medical information acquisition device) and a receiving device (e.g., a medical information storage device), efficient and accurate medical information data transmission is possible.

FIG. 9 illustrates an embodiment in which a hash value is generated based on sender identification information AE Title and receiver unique information TCP/IP number, but is not limited thereto. The cloud server 200 according to an embodiment may generate a hash value based on at least one of sender AE Title information, a sender Host Name, a sender IP address, receiver UID information, and a receiver TCP/IP number according to an embodiment.

In addition, the cloud server 200 according to another embodiment may generate a hash value based on at least one of sender AE Title information, a sender Host Name, and a sender IP address, and at least one of receiver UID information and a receiver TCP/IP number.

Meanwhile, the above-described method may be written as computer programs and may be implemented in general-use digital computers that execute the programs using a non-transitory computer readable recording medium. Furthermore, data structures used in the above-described method may be recorded in the non-transitory computer-readable recording medium via several devices. The non-transitory computer-readable recording medium may be a magnetic storage medium (e.g., ROM, RAM, USB, a floppy disk, a hard disk, etc.), or an optical reading medium (e.g., CD ROM, DVD, etc.).

It should be understood that embodiments described herein should be considered in a descriptive sense only and not for purposes of limitation. Descriptions of features or aspects within each embodiment should typically be considered as available for other similar features or aspects in other embodiments. Therefore, the scope of the disclosure is defined by the inventive concept of the appended claims.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims

1. A data transmission method comprising:
receiving medical information data comprising unique receiver information;
comparing the unique receiver information with a pre-stored mapping table to identify whether there is a medical information storage device corresponding to the unique receiver information; and
when there is the medical information storage device corresponding to the unique receiver information, transmitting the medical information data to the identified medical information storage device.

2. The data transmission method of claim 1, wherein the receiving is to receive first medical information data comprising first unique receiver information from a first medical information acquisition device,
the identifying is to compare the first unique receiver information with the pre-stored mapping table to identify whether there is a first medical information storage device corresponding to the first unique receiver information, and
the transmitting is to transmit the first medical information data to the first medical information storage device.

3. The data transmission method of claim 2, wherein the first unique receiver information is generated based on identification information of the first medical information acquisition device and unique information of the first medical information storage device.

4. The data transmission method of claim 2, wherein the receiving is to further receive second medical information data comprising second unique receiver information from the first medical information acquisition device,
the identifying is to further compare the second unique receiver information with the pre-stored mapping table to identify whether there is a second medical information storage device corresponding to the second unique receiver information, and
the transmitting is to transmit the second medical information data to the second medical information storage device.

5. The data transmission method of claim 4, wherein the second unique receiver information is generated based on identification information of the first medical information acquisition device and unique information of the second medical information storage device.

6. A non-transitory computer-readable recording medium having recorded thereon a program which, when executed by a computer, performs the method according to claim 1.

7. A cloud server comprising:
a transceiver configured to receive medical information data comprising unique receiver information, and
a processor configured to compare the unique receiver information with a pre-stored mapping table to identify whether there is a medical information storage device corresponding to the unique receiver information and to control the transceiver to transmit the medical information data to the identified medical information storage device when there is the medical information storage device corresponding to the unique receiver information.

8. The cloud server of claim 7, wherein the transceiver receives first medical information data comprising first unique receiver information from a first medical information acquisition device, and
the processor compares the first unique receiver information with the pre-stored mapping table to identify whether there is a first medical information storage device corresponding to the first unique receiver information and controls the transceiver to transmit the first medical information data to the first medical information storage device.

9. The cloud server of claim 8, wherein the first unique receiver information is generated based on identification information of the first medical information acquisition device and unique information of the first medical information storage device.

10. The cloud server of claim 8, wherein the transceiver further receives second medical information data comprising second unique receiver information from the first medical information acquisition device, and
the processor compares the second unique receiver information with the pre-stored mapping table to identify whether there is a second medical information storage device corresponding to the second unique receiver information and controls the transceiver to transmit the second medical information data to the second medical information storage device.

11. The cloud server of claim 10, wherein the second unique receiver information is generated based on identification information of the first medical information acquisition device and unique information of the second medical information storage device.

12. A data transmission system comprising:
a medical information acquisition device;
at least one medical information storage device; and
a cloud server that receives medical information data comprising unique receiver information from the medical information acquisition device, compares the unique receiver information with a pre-stored mapping table to identify whether there is a medical information storage device corresponding to the unique receiver information among the at least one medical information storage device, and transmits the medical information data to the identified medical information storage device when there is the medical information storage device corresponding to the unique receiver information.
